(19) United States
(12) Patent Application Publication
(10) Pub. No.: US 2010/0136523 A1
(43) Pub. Date: Jun. 3, 2010

(54) Molecular signature and assay for fluoroquinoline resistance in bacillus anthracis

(76) Inventors: Paul S. Keim, Flagtaff, AZ (US); Talima Pearson, Flagstaff, AZ (US); Lance B. Price, Baltimore, MD (US); Joseph D. Bush, West Lafayette, IN (US)

Correspondence Address: QUARLES & BRADY LLP, RENAISSANCE ONE, TWO NORTH CENTRAL AVENUE, PHOENIX, AZ, 85004-2391, US

(21) Appl. No.: 10/531,159

(22) PCT Filed: Oct. 14, 2003
(86) PCT No.: PCT/US03/32887

§ 371 Date: Jul. 28, 2008

Related U.S. Application Data
(60) Provisional application No. 60/417,843, filed on Oct. 11, 2002.

(51) Int. Cl. C12Q 1/68; C07H 21/04

(52) U.S. Cl. 435/6; 536/23.1; 536/24.33

(57) ABSTRACT

The preferred therapeutic for human anthrax infections are therapeutics from the fluoroquinolone class, in particular, Ciprofloxacin (CIP). This invention discloses the molecular basis for fluoroquinolone (CEP in particular) action and provides the molecular signatures which form the basis of diagnostic assays. This invention further discloses nucleotide signatures associated with CIP-resistance are useful in diagnostic tests to rapidly identify CIP resistant B. anthracis and to infer the level of resistance of these mutant strains. According to this invention, the diagnostic potential of the molecular signatures is illustrated using a primer extension assay. Further, PCR and extension primers which allow the detection of these signatures are disclosed.

### CLAIM TO DOMESTIC PRIORITY

This application claims priority to U.S. Provisional application Ser. No. 60/417,843 entitled “Molecular Signature and Assay for Fluoroquinoline Resistance in Bacillus Anthracis” filed Oct. 11, 2002, by Paul S. Keim et al., and is herein incorporated by reference in its entirety.

### FIELD OF THE INVENTION

This invention concerns generally molecular assay for Bacillus anthracis strains and more particularly, primers, methods and kits for identifying fluoroquinolone resistance in Bacillus anthracis.

### BACKGROUND OF THE INVENTION

Bacillus anthracis (B. anthracis) regularly infects livestock and wild ungulates, causing the disease anthrax. Although globally dispersed and endemic to many regions, B. anthracis shows little genetic variation between strains. Population studies using various methods of analysis including Pulse Field Gel Electrophoresis (PFGE) (Harrell et al., 1995), Single Nucleotide Polymorphisms (SNP) (Price et al., 1999; Harrell et al., 1995), Amplified Fragment Length Polymorphisms (AFLP) (Keim et al., 2000) have all found B. anthracis to be highly monomorphic.

A spore-forming zoonotic, B. anthracis occasionally infects humans, causing cutaneous, intestinal or pulmonary forms of anthrax (Friedlander, 1999). Although all three human forms are rare, the potential for using B. anthracis as a biological weapon makes development of antibiotic resistance a particularly relevant concern.

The preferred therapeutics for human anthrax infections is the fluoroquinolone, ciprofloxacin (CIP). Fluoroquinolone bactericidal action is on gyrase-DNA and topoisomerase IV-DNA complexes where drug binding causes the release of double-stranded DNA breaks (Drlica and Zhao, 1997, Piddock, 1999). Fluoroquinolone resistant mutants have amino acid changes in Quinolone Resistance Determining Regions (QRDRs) of the GyrA subunit of gyrase and the ParC subunit of topoisomerase IV. Resistance can also arise from the over-expression of multi-drug efflux pumps of the major facilitator superfamily. Low-level resistance can be acquired with a single missense mutation within a QRDR or a point mutation in the regulatory region of an efflux pump. However, high-level resistance requires a combination of mutations. The stepwise accumulation of QRDR mutations required for high-level resistance appears to follow a species-specific and predictable pathway (Ng, et al., 1996; Ferrero, et al., 1995).

Fluoroquinolone resistance, like resistance to many other antibiotics, is becoming prevalent in several clinically important species due largely to non-compliance with recommended fluoroquinolone regimens and standard regimens that are insufficient for producing inhibitory concentrations of fluoroquinolones in the soft tissue of patients (Brunner, et al., 1999). In addition to clinical sources, the use of fluoroquinolones in food-animal production has been identified as a major contributor to the emergence of fluoroquinolone resistance (Endtz et al., 1990; van den Bogaard, et al., 2000; van den Bogaard, et al., 2001).

Culturing an anthrax sample and exposing the culture to fluoroquinolene has previously been the method used to determine whether a particular strain of anthrax exhibits fluoroquinolene resistance. However, growing a bacterial culture requires a critical mass of bacterial cells in order for the culture to grow. Further, culturing is not rapid in that the bacterial culture must grow to a visible size in order to determine whether the strain is resistant.

Thus, a need exists for a means and method of reliably and rapidly determining fluoroquinolone resistant strains of B. anthracis from a small sample in order to rapidly diagnosis anthrax and develop therapeutic methods of treating anthrax, especially in developing effective tools for use in detecting and treating resistant strains of anthrax that may be used in a bioterrorism attack. Additionally, a rapid molecular-based detection method is needed to perform epidemiological studies of anthrax infections.

### SUMMARY OF THE INVENTION

It has been discovered that single nucleotide changes associated with fluoroquinoline resistance in B. anthracis mutants provide the basis of a rapid assay for detecting fluoroquinoline resistant, species directly from B. anthracis DNA. Cipro-resistant mutants have been isolated and characteristic SNP sequences have been identified. Primers and primer pairs are presented for assaying these SNP sequences in amplification assays, preferably multiplex. Kits useful for multiplexing sample DNA from B. anthracis strains are given.

Isolated oligonucleotides are provided comprising at least 12 consecutive nucleotides of a nucleic acid sequence selected from the group of consisting of: SEQ ID NO: 1; SEQ NO: 2; SEQ ID NO:3; SEQ ID NO: 4; SEQ ID NO: 5; SEQ ID NO: 6; SEQ ID NO: 7; SEQ ID NO: 8; SEQ ID NO: 9; SEQ ID NO: 10; SEQ ID NO: 11; SEQ ID NO: 12; SEQ ID NO: 13; SEQ ID NO: 14; SEQ ID NO: 15; SEQ ID NO: 16; SEQ ID NO: 17; SEQ ID NO: 18 SEQ ID NO: 19; SEQ ID NO: 20; SEQ ID NO: 21; SEQ ID NO: 22; SEQ ID NO: 23; SEQ ID NO: 24; SEQ ID NO: 25; SEQ ID NO: 26; SEQ ID NO: 27; SEQ ID NO: 28; SEQ ID NO: 29; SEQ ID NO: 30; SEQ ID NO: 31; SEQ ID NO: 32; SEQ ID NO: 33; SEQ ID NO: 34; SEQ ID NO: 35; SEQ ID NO: 36; SEQ ID NO: 37; SEQ ID NO: 38; SEQ ID NO: 39; SEQ ID NO: 40; SEQ ID NO: 41; SEQ ID NO: 42; SEQ ID NO:43; SEQ ID NO: 44; SEQ ID NO: 45; SEQ ID NO: 46; SEQ ID NO: 47; SEQ ID NO: 48; SEQ ID NO: 49; SEQ ID NO: 50; SEQ ID NO: 51; SEQ ID NO: 52; and SEQ ID NO: 53; wherein the oligonucleotide is capable of binding selectively to DNA indicating fluoroquinoline resistance in Bacillus anthracis.

In certain preferred embodiments of the invention the oligonucleotide are immobilized on a solid surface, a chromatographic surface, e.g., or a nanometric scale diagnostic plate. In other preferred embodiments of the invention the nucleotides further comprise an observable marker, most preferably a fluorescent label or a radioactive group.

Primer pairs selected from the group of oligonucleotide pairs consisting of: SEQ ID NO: 1 and SEQ ID NO: 2; SEQ ID NO: 3 and SEQ ED NO: 4; SEQ ID NO: 5 and SEQ ID NO: 6; SEQ ID NO: 7 and SEQ ID NO: 8; SEQ ID NO: 9 and SEQ ID NO: 10; SEQ ID NO: 11 and SEQ ID NO: 12; SEQ ID NO: 13 and SEQ ID NO: 14; SEQ ID NO: 15 and SEQ ID NO: 16; SEQ ID NO: 17 and SEQ ID NO: 18; SEQ ID NO: 19 and SEQ ID NO: 20; SEQ ID NO: 21 and SEQ ID NO: 22; SEQ ID NO: 23 and SEQ ID NO: 24; SEQ ID NO: 25 and SEQ ID NO: 26; SEQ ID NO: 27 and SEQ ID NO: 28; SEQ ID NO: 29 and SEQ ID NO: 30; SEQ ID NO: 31 and SEQ ID NO: 32; SEQ ID NO: 33 and SEQ ID NO: 34; SEQ ID NO: 35 and SEQ ID NO: 36; SEQ ID NO: 37 and SEQ ID NO: 38; and SEQ ID NO: 39 and SEQ ID NO: 40 are presented wherein the pair of oligonucleotide primers is capable of binding selectively to DNA indicating fluoroquinoline resistance in Bacillus anthracis.

Internal oligonucleotide primer selected from the group consisting of SEQ ID NO: 41; SEQ ID NO: 42; SEQ ID NO:43; SEQ ID NO: 44; SEQ ID NO: 45; SEQ ID NO: 46; SEQ ID NO: 47; SEQ ID NO: 48; SEQ ID NO: 49; SEQ ID NO: 50; SEQ ID NO: 51; SEQ ID NO: 52; and SEQ ID NO: 53 are presented wherein the primer is capable of detecting a single nucleotide polymorphism, wherein the single nucleotide polymorphism is characteristic of fluoroquinoline resistance in Bacillus anthracis.

Single base extension (SBE) primers comprising the internal oligonucleotide primers and a polypolynucleotide tails for use in the amplification and separation of SNP in a PCR instrument wherein the SBE primers provide customizied amplicon lengths to aid electrophoretic separation of the amplicons.

In an important aspect of the invention methods are presented for detecting fluoroquinolone resistant B. anthracis strains by detecting the presence or absence of a plurality of selected target DNA sequences associated with fluoroquinolone resistance in Bacillus anthracis.

Certain preferred methods for detecting a fluoroquinoline resistant strain of Bacillus anthracis comprise the steps of:
i. providing a DNA sample from a Bacillus anthracis strain;
ii. providing one or more primer pairs selected from the group of oligonucleotide pairs consisting of: SEQ ID NO: 1 and SEQ ID NO: 2; SEQ ID NO:3 and SEQ ID NO: 4; SEQ ID NO: 5 and SEQ ID NO: 6; SEQ ID NO: 7 and SEQ ID NO: 8; SEQ ID NO: 9 and SEQ ID NO: 10; SEQ ID NO: 11 and SEQ ID NO: 12; SEQ ID NO: 13 and SEQ ID NO: 14; SEQ ID NO: 15 and SEQ ID NO: 16; SEQ ID NO: 17 and SEQ ID NO: 18; SEQ ID NO: 19 and SEQ ID NO: 20; SEQ ID NO: 21 and SEQ ID NO: 22; SEQ ID NO: 23 and SEQ ID NO: 24; SEQ ID NO: 25 and SEQ ID NO: 26; SEQ ID NO: 27 and SEQ ID NO: 28; SEQ ID NO: 29 and SEQ ID NO: 30; SEQ ID NO: 31 and SEQ ID NO: 32; SEQ ID NO: 33 and SEQ ID NO: 34; SEQ ID NO: 35 and SEQ ID NO: 36; SEQ ID NO: 37 and SEQ ID NO: 38; and SEQ ID NO: 39 and SEQ ID NO: 40; wherein the pair of oligonucleotide primers is capable of binding selectively to DNA indicating fluoroquinoline resistance in Bacillus anthracis;
iii. amplifying said DNA with one or more said primer pairs; and
iv. comparing the results of said multiplexing step with results of amplification of DNA from known fluoroquinoline resistant strains.

Preferrably amplification of DNA is by multiplexing with one or more suitable primer pairs. Other preferred embodiments of the method for detecting fluoroquinoline resistance in Bacillus anthracis comprises the steps of:
i. providing a DNA sample from Bacillus anthracis;
ii. providing one or more isolated oligonucleotide comprising at least 12 consecutive nucleotides of a nucleic acid sequence selected from the group of consisting of: SEQ ID NO: 1; SEQ ID NO: 2; SEQ ID NO:3; SEQ ID NO: 4; SEQ ID NO: 5; SEQ ID NO: 6; SEQ ID NO: 7; SEQ ID NO: 8; SEQ ID NO: 9; SEQ ID NO: 10; SEQ ID NO: 11; SEQ ID NO: 12; SEQ ID NO: 13; SEQ ID NO: 14; SEQ ID NO: 15; SEQ ID NO: 16; SEQ ID NO: 17; SEQ ID NO: 18, SEQ ID NO: 19; SEQ ID NO: 20; SEQ ID NO: 21; SEQ ID NO: 22; SEQ ID NO: 23; SEQ ID NO: 24; SEQ ID NO: 25; SEQ ID NO: 26; SEQ ID NO: 27; SEQ ID NO: 28; SEQ ID NO: 29; SEQ ID NO: 30; SEQ ID NO: 31; SEQ ID NO: 32; SEQ ID NO: 33; SEQ ID NO: 34; SEQ ID NO: 35; SEQ ID NO: 36; SEQ ID NO: 37; SEQ ID NO: 38; SEQ ID NO: 39; SEQ ID NO: 40 SEQ ID NO: 41; SEQ ID NO: 42; SEQ ID NO:43; SEQ ID NO: 44; SEQ ID NO: 45; SEQ ID NO: 46; SEQ ID NO: 47; SEQ ID NO: 48; SEQ ID NO: 49; SEQ ID NO: 50; SEQ ID NO: 51; SEQ ID NO: 52; and SEQ ID NO: 53; wherein the oligonucleotide is capable of binding selectively to DNA indicating fluoroquinoline resistance in Bacillus anthracis.
iii. combining said oligonucleotides and said DNA under conditions whereby said DNA binds to said oligonucleotides; and
iv. detecting the presence or absence of bound oligonucleotides;
wherein the presence of bound oligonucleotide indicates a fluoroquinoline resistant B. anthracis strain.

Preferably in this preferred method the oligonucleotides comprise an observable marker, most preferably fluorescent or radioactive group. Other preferred methods of the present invention for detecting a fluoroquinoline resistant strain of Bacillus anthracis comprise the steps of:
i. providing a DNA sample from a Bacillus anthracis strain;
ii. providing one or more primer pairs selected from:
   a) A pair of oligonucleotide primers selected from the group of oligonucleotide pairs consisting of: SEQ ID NO: 1 and SEQ ID NO: 2; SEQ ID NO:3 and SEQ ID NO: 4; SEQ ID NO: 5 and SEQ ID NO: 6; SEQ ID NO: 7 and SEQ ID NO: 8; SEQ ID NO: 9 and SEQ ID NO: 10; SEQ ID NO: 11 and SEQ ID NO: 12; SEQ ID NO: 13 and SEQ ID NO: 14; SEQ ID NO: 15 and SEQ ID NO: 16; SEQ ID NO: 17 and SEQ ID NO: 18; SEQ ID NO: 19 and SEQ ID NO: 20; SEQ ID NO: 21 and SEQ ID NO: 22; SEQ ID NO: 23 and SEQ ID NO: 24; SEQ ID NO: 25 and SEQ ID NO: 26; SEQ ID NO: 27 and SEQ ID NO: 28; SEQ ID NO: 29 and SEQ ID NO: 30; SEQ ID NO: 31 and SEQ ID NO: 32; SEQ ID NO: 33 and SEQ ID NO: 34; SEQ ID NO; 35 and SEQ ID NO: 36; SEQ ID NO: 37 and SEQ ID NO: 38; and SEQ ID NO: 39 and SEQ ID NO: 40; wherein the pair of oligonucleotide primers is capable of binding selectively to DNA indicating fluoroquinoline resistance in Bacillus anthracis;
   b) an internal oligonucleotide primer selected from the group consisting of SEQ ID NO: 41; SEQ ID NO: 42; SEQ ID NO:43; SEQ ID NO: 44; SEQ ID NO: 45; SEQ ID NO: 46; SEQ ID NO: 47; SEQ ID NO: 48; SEQ ID NO: 49; SEQ ID NO: 50; SEQ ID NO: 51; SEQ ID NO: 52; and SEQ ID NO: 53, wherein the primer is capable of detecting a single nucleotide polymorphism, wherein the single nucleotide polymorphism is characteristic of fluoroquinoline resistance in Bacillus anthracis; or
   c) combinations thereof;
iii. amplifying said DNA with one or more said primers, said primers preferably comprising an observable marker, most preferably a fluorescent or radioactive group; and
iv. comparing the results of said amplification step with results of amplification of a known fluoroquinoline resistant B. anthracis strain with said primers.

In preferred methods, single base extension (SBE) primers (Table 3) comprising an internal oligonucleotide primer and further comprising polynucleotide tails for use in the amplification and separation of SNP in a PCR instrument wherein the SBE primers provide customizied amplicon lengths to aid electrophoretic separation. In another important aspect of the present invention, kits are provided for detecting fluoroquinolones resistant B. anthracis by thermal recycling amplification, preferably multiplexing. Preferred kits for the detection of fluoroquinoline resistance in Bacillus anthracis comprise one or more pairs of oligonucleotide primers pairs selected from the group of oligonucleotide pairs consisting of: SEQ ID NO: 1 and SEQ ID NO: 2; SEQ ID NO:3 and SEQ ID NO: 4; SEQ ID NO: 5 and SEQ ID NO: 6; SEQ ID NO: 7 and SEQ ID NO: 8; SEQ ID NO: 9 and SEQ ID NO: 10; SEQ ID NO: 11 and SEQ ID NO: 12; SEQ ID NO: 13 and SEQ ID NO: 14; SEQ ID NO: 15 and SEQ ID NO: 16; SEQ ID NO: 17 and SEQ ID NO: 18; SEQ ID NO: 19 and SEQ ID NO: 20; SEQ ID NO: 21 and SEQ ID NO: 22; SEQ ID NO: 23 and SEQ ID NO: 24; SEQ ID NO: 25 and SEQ ID NO: 26; SEQ ID NO: 27 and SEQ ID NO: 28; SEQ ID NO: 29 and SEQ ID NO: 30; SEQ ID NO: 31 and SEQ ID NO: 32; SEQ ID NO: 33 and SEQ ID NO: 34; SEQ ID NO: 35 and SEQ ID NO: 36; SEQ ID NO: 37 and SEQ ID NO: 38; SEQ ID NO: 39 and SEQ ID NO: 40; wherein the pair of oligonucleotide primers is capable of causing amplification of a product that indicates fluoroquinoline resistance in B. anthracis.

Most preferably the kits of the present invention further comprise tnps, most preferably labeled, for example, ATP, TTP, GTP, CTP and UTP, and taq polymerase, salts and buffer suitable for causing amplification of said DNA in a PCR instrument. Certain other kits comprise one or more oligonucleotide primer selected from the group consisting of: SEQ ID NO: 41; SEQ ID NO: 42; SEQ ID NO:43; SEQ ID NO: 44; SEQ ID NO: 45; SEQ ID NO: 46; SEQ ID NO: 47; SEQ ID NO: 48; SEQ ID NO: 49; SEQ ID NO: 50; SEQ ID NO: 51; SEQ ID NO: 52; and SEQ ID NO: 53, wherein the primers are capable of detecting a single nucleotide polymorphism, wherein the single nucleotide polymorphism is characteristic of fluoroquinoline resistance in Bacillus anthracis. Most preferably the primers are labeled with an observable marker, a fluorescent or radioactive group. In preferred embodiments, the kits further comprise salts and buffer suitable for causing binding of DNA and primers.

### BRIEF DESCRIPTION OF THE DRAWINGS

FIG. 1 illustrates mutant sensitivity to different Ciprofloxacin concentrations. The wild type progenitor strain (A) and three sequential stepwise mutant strains (B-D) are evaluated for Ciprofloxacin sensitivity using E-strips. The stepwise mutant strains are (B) S1-1, (C) S2-3 and (D) S3-1. The spontaneous stepwise mutation rates in changes per generation shown in the arrows.

FIG. 2 illustrates SNP assays for mutational changes associated with CIP resistance. An illustration of an ABI377 gel image is shown with nine SNP loci across seven B. anthracis strains (wild type, two step 1 mutants, two step 2 mutants, and two step 3 mutants). In addition, one electropherogram of the wild type genotype generated on a capillary electrophoresis instrument (AB3100) is shown to illustrate the assay's flexibility across diagnostic platforms. SNP of mutant genotypes are: S1-1: gyrA254(R) C→T; S1-2: gyrA265(R) G→A; S2-1: gyrA254(R) C→T & parC242 C→T; S2-2: gyrA254(R) C→T & parC242 C→A; S3-1: gyrA254(R) C→T &→265(R) G→A & parC242 C→T; S3-2: gyrA254(R) C→T & gyrA266(R) A→C & parC242 C→T.

### DETAILED DESCRIPTION

The present invention discloses a molecular assay for screening B. anthracis for single nucleotide polymorphism (SNPs) associated with Ciprofloxacin (CIP) resistance. This diagnostic approach provides a rapid screening of B. anthracis samples for CIP resistance in situations where culturing the sample is not possible. It is anticipated that in the event of any bioterrorist activity, this rapid assay will make possible the early detection of malicious spread of anthrax.

A study of multiple mutant B. anthracis strains showed that the primary target of CIP in wild-type B. anthracis is GyrA, the secondary target is ParC and the tertiary targets are yet to be fully determined. The target order of CIP appears to be determined by the amino acid residues of the Gyrase and Toposiomerase IV subunit QRDRs.

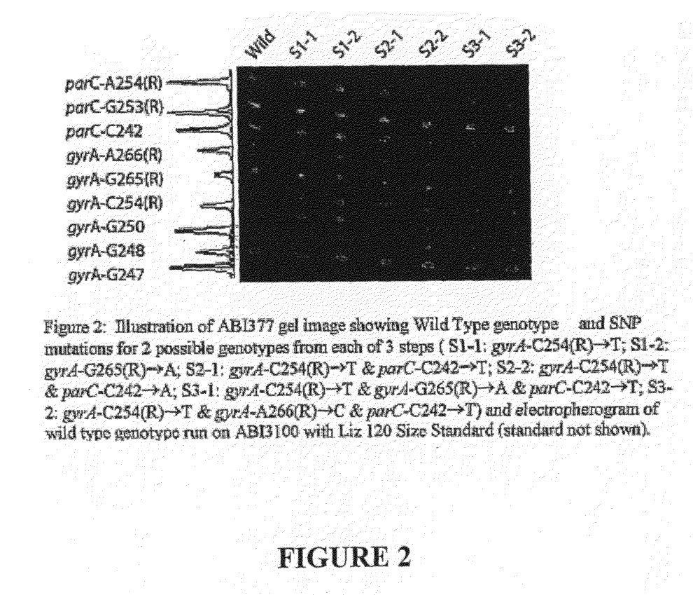

Assays are presented for determining fluoroquinolones resistant B. anthracis based on the mutational status of the six gyrA and three parC nucleotides. Primers for multiplexing to amplify nine loci are disclosed in Table 3 and FIG. 2. These nine loci represent the most common mutants and may be quickly assayed using the present methods. Because these nine mutations play a critically important role in determining the level of CIP resistance, SNP information may be used in developing an appropriate antibiotic treatment strategy at an early stage of an outbreak. A newly acquired strain could be genotyped in just a few hours.

Strains of CIP resistant B. anthracis that arise either by misuse of antibiotics or malevolence, can be rapidly genotyped using the disclosed SNP assay This assay was developed using the SNaPshot™ technology (ABI PRISMT™ Applied BioSystems Inc.) but other SNP assays known in the art are available and may be used. In the present assay, the mutational status of the six gyrA and three parC nucleotides is easily observed in a single lane on an ABI377 or ABI3100.

The following definitions are used herein:

“Polymerase chain reaction” or “PCR” a technique in which cycles of denaturation, annealing with primer, and extension with DNA polymerase are used to amplify the number of copies of a target DNA sequence by approximately 106 times or more. The polymerase chain reaction process for amplifying nucleic acid is disclosed in U.S. Pat. Nos. 4,683,195 and 4,683,202, which are incorporated herein by reference.

“Primer” a single-stranded oligonucleotide or DNA fragment which hybridizes with a DNA strand of a locus in such a manner that the 3′ terminus of the primer may act as a site of polymerization using a DNA polymerase enzyme.

“Primer pair” two primers including, primer 1 that hybridizes to a single strand at one end of the DNA sequence to be amplified and primer 2 that hybridizes with the other end on the complementary strand of the DNA sequence to be amplified.

“Primer site”: the area of the target DNA to which a primer hybridizes.

“Multiplexing” is an assay system for simultaneous, multiple determinations in a single assay process in a thermocycling instrument (PCR). A process to implement such a capability in a process is a “multiplexed assay.” Systems containing several loci are called multiplex systems described, for example, in U.S. Pat. No. 6,479,235 to Schumm, et al., U.S. Pat. No. 6,270,973 to Lewis, et al. and U.S. Pat. No. 6,449,562 to Chandler, et al.

“Isolated nucleic acid” is a nucleic acid which may or may not be identical to that of a naturally occurring nucleic acid. When “isolated nucleic acid” is used to describe a primer, the nucleic acid is not identical to the structure of a naturally occurring nucleic acid spanning at least the length of a gene. The primers herein have been designed to bind to both sequences flanking. Certain primers also may bind internally to the DNA sequence of interest. It is to be understood that primer sequences containing insertions or deletions in these disclosed sequences that do not impair the binding of the primers to these flanking sequences are also intended to be incorporated into the present invention.

Method for Rapid Assay of B. anthracis for Detection of Fluoroquinolones-Resistant Strains.

DNA extraction. DNA was extracted as described in Keim et. al., 2000. Briefly, DNA was extracted from each resistant mutant by suspending ˜1 mg of cellular material from blood agar plates in 150 μl of heat-soak buffer (10 mM Tris-HCl, 1 mM EDTA, pH 8.0) and heating to 85° C. for 30 min. Cellular debris was pelleted by centrifugation and the supernatant was used as template in PCR reactions.

PCR amplification. All primers (Table 1) were designed from the incomplete B. anthracis genome sequence generally provided by The Institute for Genomic Research, Rockville, Md., USA. PCR products were amplified in 50 μl PCR reactions and prepared as follows: 1×PCR buffer (20 mM Tris pH 8.4, 50 mM KCl) (Gibco/BRL, Bethesda, Md., USA), 0.10 mM DNTPs, 2 mM MgCl₂, 2 μl heat-soak supernatant as template, 0.04 U/μl Tag DNA Polymerase (Gibco/BRL, Bethesda, Md., USA), 0.2 μM forward and reverse primers, adjusted to 50 μl with filtered (0.2 μm) 17.8 mOhm E-pure water. Reactions were heated to 94° C. for 5 min, then subjected to 35 cycles of 20 s at 94° C., 20 s at 60° C. and 20 s at 72° C. This was followed by heating to 72° C. for 5 min to complete primer extension. PCR products were quantified on. EtBr stained 1.5% Synergel™ (Diversified Biotech, Boston, Mass., USA)/0.7% agarose (Gibco/BRL, Bethesda, Md., USA). Quantified PCR products were sequenced as follows.

DNA sequencing. PCR products were diluted 1:5 in water and sequenced on an ABI377 fluorescent sequencer using the ABI PRISM® Ready Reaction BigDye™ Terminator Cycle Sequencing Kit (both from Perkin-Elmer/Applied Biosystems Inc., Foster City, Calif., USA). When necessary, contiguous gene sequences were prepared from the individual sequences using SeqMan™ software (DNASTAR, inc., Madison, Wis., USA). Contiguous Sequences were aligned with the wild-type sequences using MegAlign™ software (DNASTAR, inc., Madison, Wis., USA).

SNP multiplex assay. A single nucleotide polymorphism (SNP) assay was developed to rapidly identify the nine observed resistance mutations (Table 2). Flanking primers (Table 3) were designed to amplify bases 203-323 of gyrA (122 by product) and bases 175-320 of parC (146 by product). PCR products were amplified in 10 μl singleplex or duplex PCRs with final concentrations of 1×PCR Buffer (above), 3 mM MgCl₂, 0.1 mM dNTPs, forward and reverse primer pairs (0.1 μM gyrA primers/0.4 μM parC primers), 1 U Platinum® Taq DNA Polymerase (Gibco/BRL, Bethesda, Md., USA), and 1 μl heat-soak supernatant as template. Reactions were heated to 94° C. for 5 min, then subjected to 30 cycles of 20 s at 94° C., 30 s at 60° C. and 30 s at 72° C. The remainder of the procedure was carried out according to methods known in the art. Preferred instructions are given in the ABI PRISM® SNaPshot™ Multiplex Kit and run on an AB3100 genetic analyzer (Applied Biosystems, Foster City, Calif., USA). Single base extension (SBE) primers (Table 3) were designed with polynucleotide tails (poly-Cs and single As) to customize amplicon lengths to 4-bp intervals such that when separated electrophoretically, the six gyrA SNPs were detected in the 5′ to 3′ order in which mutations are found, followed by the three parC SNPs (also in 5′ to 3′ order of occurrence). Since primers gyrA265 and gyrA254 overlapped 1 and 2 SNP loci respectively, they were designed with degenerate base pairs at sites 266 and 265 to allow annealing on templates with step 3 mutations. Despite primer degeneracy, amplification of gyrA265 in the 13-primer multiplex was weak on S3-2 mutants. This locus was therefore targeted individually by performing a second SBE containing only the two gyrA265 primers when a template was suspected to be an S3-2 mutant. The order of SBE products enabled multiplexing of SBE PCRs and facilitated scoring, eliminating the need for a size standard. Therefore, this assay can be performed on a 4-dye ABI377 if a 5-dye capillary machine is not available.

TABLE 1
Primers Used

| | | | Gene/ |
|---|---|---|---|
| SEQ ID # | Name | Sequence (5′ [symbol] 3′) | Region |
| SEQ ID #1 | ParC QRDR F | GTGTTAGGTGACCGCTTTG | parC/ |
| | | CACGTTATAGTAAATA | QRDR |
| SEQ ID #2 | ParC QRDR R | GTAAAACAACCGGTTCTTC | parC/ |
| | | ACTCGTATCATC | QRDR |
| SEQ ID #3 | GyraA QRDR F | ACGTATTAATTCCATAGAG | gyrA/ |
| | | ATTTTAGACATTCTTGCTT | QRDR |
| | | CTGTAT | |
| SEQ ID #4 | GyraA QRDR R | CATTTTTAGATTACGCAAT | gyrA/ |
| | | GAGTGTTATCGTATCTCG | QRDR |
| SEQ ID #5 | BA ParC aF1 | GGTACGACAGTTGCCCAAA | parC |
| | | ATGATGGTT | |
| SEQ ID #6 | BA ParC aR1 | CAAGCGGAAGCAATTGTAT | parC |
| | | CCT | |
| SEQ ID #7 | BA ParC bF1 | CGCGTCGATCATCACTATA | parC |
| | | TGTTTTCTTAACTCTC | |
| SEQ ID #8 | BA ParC bR1 | ATTATTATTCGCGGGAAAG | parC |
| | | CAGAGGTTGA | |
| SEQ ID #9 | BA ParC cF1 | GTCTCATCACGTACTTCAG | parC |
| | | CAATGCCATCT | |
| SEQ ID #10 | BA ParC cR1 | TCGGCTAAAACAGTCGGTA | parC |
| | | ACGTTATTGGTAA | |
| SEQ ID #11 | BA ParC-E F1 | CGGATCCCCGTCAACAC | parC & |
| | | | parE |
| SEQ ID #12 | BA ParC-E R1 | CGGATCAATTATGGGAAAC | parC & |
| | | AACGATGAATC | parE |
| SEQ ID #13 | BA ParE aF1 | AAGCGGGAGGTCATGAAAC | parE |
| | | TTCTCTGC | |
| SEQ ID #14 | BA ParE aR1 | AGTGGTAAGTTAACACCCG | parE |
| | | CACAATCACG | |
| SEQ ID #15 | BA ParE bF1 | CCCTTGTTTCGCAGAACCA | parE |
| | | C | |
| SEQ ID #16 | BA ParE bRa | TTGAAGCTTTCGTTTCCTA | parE |
| | | T | |
| SEQ ID #17 | BA ParE cF1 | CTAATTCTGCTTCAATCCC | parE |
| | | ATTTTGTTCACC | |
| SEQ ID #18 | BA ParE cRa | RAGCGTTATAGATAAAGGG | parE |
| | | CGAGGAATG | |
| SEQ ID #19 | BA ParE dF1 | ACACCGCCATTTTCAAAGC | parE |
| | | GTTGTTC | |
| SEQ ID #20 | BA ParE dR1 | GATTTTGGATTAGGAAAGG | parE |
| | | GGCAAGGAGTT | |
| SEQ ID #21 | BA GyrB aF1 | CGACGGAATTGAACACGAA | gyrB |
| | | ACA | |
| SEQ ID #22 | BA GyrB aR1 | TACAGATGCCCCAACACC | gyrB |
| SEQ ID #23 | BA GyrB bF1 | ATGGGACGTCCTGCTGTAG | gyrB |
| | | AAGTTATTATGACC | |
| SEQ ID #24 | BA GyrB bR1 | AGTTAAACCTTCACGAACG | gyrB |
| | | TCCTCACCAGTTA | |
| SEQ ID #25 | BA GyrB cF1 | ACGTATGAAGGTGGAACAC | gyrB |
| | | ATGAAGTAGGGTTTA | |
| SEQ ID #26 | BA GyrB cR1 | GCTTTCTCAATATCAAAAT | gyrB |
| | | CTCCGCCAATGT | |
| SEQ ID #27 | BA GyrB dF1 | CGTCACTTCCAAGCGATTT | gyrB |
| | | TACCACTGAA | |
| SEQ ID #28 | BA GyrB dR1 | ACCTCCTCTTACATTTCCG | gyrB |
| | | TTACACATACATTGATTTA | |
| | | T | |
| SEQ ID #29 | BA GyrB-A F1 | GGGGGATAAAGTAGAGCCA | gyrB & |
| | | CGTCGTAACT | gyrA |
| SEQ ID #30 | BA GyrB-A R1 | AGGAAAACGCGCTGGTAAC | gyrB & |
| | | A | gyrA |
| SEQ ID #31 | BA GyrA aF1 | CAGCAATGCGTVATACAGA | gyrA |
| | | AGCAAGAATGTC | |
| SEQ ID #32 | BA Gyra aR1 | TGCCTTTTCAAGTTCATAA | gyrA |
| | | GCAGTA | |
| SEQ ID #33 | BA GyrA bF1 | GGAAGTACGTCGTGATGCC | gyrA |
| | | AATGCTAATG | |
| SEQ ID #34 | BA GyrA bR1 | ATACCTTTCGCTGTACGAC | gyrA |
| | | TATACTCTGGGATTTC | |
| SEQ ID #35 | BA GyrA cF1 | CAGAACAAAACATCGCCAT | gyrA |
| | | TACGTTAACTCATAA | |
| SEQ ID #36 | BA GyrA cR1 | AGAGATTTGATCAACTGGC | gyrA |
| | | ATACGAATAATAACACC | |

TABLE 2
Identity of Ciprofloccion Resistant Signatures

| | MIC | gyrA mutation | | parC mutation | |
|---|---|---|---|---|---|
| Mutant | [CIP μg/ml] | Δ Nucleotide | Δ Amino Acid | Δ Nucleotide | Δ Amino Acid |

| | | | | | |
|---|---|---|---|---|---|
| S1-1 | 0.38 | C254 [symbol] *T | S85 [symbol] *L | — | — |
| S1-2 | 0,38 | G265 [symbol] A | E89 [symbol] K | — | — |
| S1-3 | ≧0.25 | G248 [symbol] A | G83 [symbol] D | — | — |
| S1-4 | ≧0.25 | G250 [symbol] A | D84 [symbol] N | — | — |
| S1-5 | ≧0.25 | G247 [symbol] T | G83 [symbol] C | — | — |
| S2-1 | 12 | C254 [symbol] T | S85 [symbol] L | C242 [symbol] T | S81 [symbol] F |
| S2-2 | 4 | C254 [symbol] T | S85 [symbol] L | C242 [symbol] A | S81 [symbol] Y |
| S2-3 | ≧1.5 | C254 [symbol] T | S85 [symbol] L | G253 [symbol] A | E85 [symbol] K |
| S2-4 | ≧1.5 | C254 [symbol] T | S85 [symbol] L | A254 [symbol] G | E85 [symbol] G |
| S3-1 | 64 | C254 [symbol] T | S85 [symbol] L | C242 [symbol] T | S81 [symbol] F |
| | | G265 [symbol] A | E89 [symbol] K | | |
| S3-2 | 64 | G254 [symbol] T | S85 [symbol] L | C242 [symbol] T | S81 [symbol] F |
| | | A266 [symbol] C | E89 [symbol] A | | |
| S3-x | 16, 24, | C254 [symbol] T | S85 [symbol] L | C242 [symbol] T | S81 [symbol] F |
| | 48, 64 | | | | |

TABLE 3
External and SBE primers used in the SNP assay

| | | | | |
|---|---|---|---|---|
| | | | Product | |
| SEQ ID # | Name | Sequence (5′→3′) | Size | SNP |

External primers

| | | | | |
|---|---|---|---|---|
| SEQ ID #37 | BAgyrA01F_flanking | TCAGCACGTATTGTTGGTGAAG | 122 | — |
| SEQ ID #38 | BAgyrA01R_flanking | TGCCCATCAACAAGCATATAAC | 122 | — |
| SEQ ID #39 | BAparC02F_flanking | AAAGCGTTCCGTAAGTCGG | 145 | — |
| SEQ ID #40 | BAparC02R_flanking | TTATTACCATGCATCTCAACT | 145 | — |
| | | AAAAC | | |
| SBE primers | | | | |
| SEQ ID #41 | BAgyrASNP247F_ | ATCGGTAAGTATCACCCTCAT | 22 | G/T |
| | internal | | | |
| SEQ ID #42 | BAgyrASNP248F_ | CccccCGGTAAGTATCACCCTC | 26 | G/A |
| | internal | ATG | | |
| SEQ ID #43 | BAgyrASNP250F_ | cccccccGGTAAGTATCACCCTC | 30 | G/A |
| | internal | ATGGT | | |
| SEQ ID #44 | BAgyrASNP254R_ | cccccccccccccCCATCGTTTCAT | 34 | C/T^{†} |
| | internal | AAACAGCT | | |
| SEQ ID #45 | BAgyrASNP254R(G)_ | cccccccccccccCCATCGTTGCAT | 34 | C/T^{†} |
| | internal | AAACAGCT | | |
| SEQ ID #46 | BAgyrASNP254R(T)_ | cccccccccccccCCATCGTTTTAT | 34 | C/T^{†} |
| | internal | AAACAGCT | | |
| SEQ ID #47 | BAgyrASNP254R(GT)_ | cccccccccccccCCATCGTTGTAT | 34 | C/T^{†} |
| | internal | AAACAGCT | | |
| SEQ ID #48 | BAgyrASNP265R_ | cccccccccccccccccccGCCATACG | 38 | G/A^{†} |
| | internal | TACCATCGTTT | | |
| SEQ ID #49 | BAgyrASNP265R(G)_ | cccccccccccccccccccGCCATACG | 38 | G/A^{†} |
| | internal | TACCATCGTTG | | |
| SEQ ID #50 | BAgyrASNP266R_ | ccccccccccccccccccccccccCGCCA | 42 | A/C^{†} |
| | internal | TACGTACCATCGTT | | |
| SEQ ID #51 | BAparCSNP242F_ | ccccccccccccccccccccccccccccccc | 47 | C/T/A |
| | internal | CACCCGCACGGTGATT | | |
| SEQ ID #52 | BAparCSNP253R_ | ccccccccccccccccccccccccccccGA | 51 | G/A^{†} |
| | internal | CTTAAACGTACCATCGCTT | | |
| SEQ ID #53 | BAparCSNP254R_ | cccccccccccccccccccccccccccccccc | 54 | A/G^{†} |
| | internal | cAGCGATGGTACGTTTAAGTC | | |

^{†}SNPs will be detected as reverse complements in the SNaPshot ™ assay when reverse SBE primers are used.

Selection of Mutant B. anthracis Strains and Identification of Fluoroquinolones-Resistant Sites

Bacterial strains. Selections were performed on the non-virulent, pX01-/pX02-, Ames strain of B. anthracis (Ivins et al., 1986). All DNA samples used for the diversity study came from our B. anthracis DNA collection (Keim, et al., 2000). B. anthrocis strains were selected sequentially at increasing CTP concentrations to produce a resulting stepwise accumulation of mutations. Mutant strains were isolated with MICs as high as 64 μg CIP/ml (1000-fold higher than wild-type) These results are given in Table 2. The accumulation of mutations occurred in a distinctive and ordered manner. First level mutants, selected on 0.25 μg CIP/ml, developed at a rate of 6.6×10⁻¹⁰ and had one of five mutations within the gyrA QRDR (Table 2). A disproportionate number (71%) of these mutants possessed the C254→T missense mutation in gyrA (Table 2). The level of resistance conferred by this mutation was similar to that of other S1 mutations. Since this mutation provided no selective advantage over the other S1 mutations, it is reasonable to call the C254 nucleotide of B. anthracis gyrA a mutational hotspot. Second level mutants, selected on 1.5 μg CIP/ml, developed at a rate of 1.0×10⁻⁸ and possessed one of four mutations within the parC QRDR (Table 2). As with the S1 mutants one S2 genotype, C242→T, was overrepresented (71%) (Table 2). While it is likely that the C242 nuelcotide of parC represents another mutational hotspot, the overrepresentation could also be a result of the disproportionate level of resistance conferred to the strain by the mutation (Table 2). Third level mutants, selected on 24 μg CIP/ml, developed at a rate of 4.8×10⁻¹⁰. Two third-level mutants were identified with novel mutations within the gyrA QRDR (Table 2). However, the other 21 third-level mutants had no additional alterations in either the gyrA or parC QRDRs. Potential QRDRs in gyrB and parE were also sequenced from these strains and revealed no additional mutations in these regions. The targeted stepwise accumulation of mutations (S1 gyrA→S2 parC→S3 gyrA/?) give further evidence to support the hypothesis that particular fluoroquinolones have different primary topoisomerase targets within various bacterial species (Ferrero et al., 1995; Ng et al., 1996; Pan and Fisher et al., 1998).

B. anthracis has the ability to develop a number of different missense mutations that enable it to grow in the presence of CIP. The stepwise phenotypic rates at which B. anthracis develops resistance to CIP (4.8×10⁻¹⁰ to 1.0×10⁻⁸) are similar to those reported for fluoroquinolone resistance in other species. The rarity of human anthrax cases and the carcass-dependent transmission cycle of this pathogen make the development and spread of CIP resistant B. anthracis through patient non-compliance unlikely. However, the agricultural practice of antimicrobial growth promotion does have this potential outcome. CIP regimens targeted at serum and tissue concentrations of ≧0.38 μg CIP/ml would reduce the chances for developing CIP resistant B. anthracis by requiring the statistically unlikely event (6.6×10⁻¹⁸) of a bacterium to develop advantageous mutations in the gyrA and parC QRDRs simultaneously. The serum and tissue concentrations resulting from the low-level feeding of antibiotics as antimicrobial growth promoters in livestock would not reach the MPC and likely fall short of the MIC for wild-type B. anthracis. Therefore, this practice could present a potential risk for the development of resistant strains, particularly in those livestock regions to which B. anthracis is endemic.

Diversity Study. The QRDRs of gyrA and parC were sequenced from eight major diversity groups and analyzed for point mutations as described below. The eight strains, 3 (74-42C-8), 25 (14185), 39 (46), 45 (2B80), 62 (Oct-321), 77 (Vollum), 80

Stepwise mutant selection. B. anthracis Ames −/− strain was taken from a frozen stock, streaked onto blood agar plates and grown overnight at 35° C. Cells from isolated colonies were used to inoculate culture tubes containing 5 ml of Mueller-Hinton broth. Cultures were incubated overnight at 37° C. in a G24

Environmental Incubator Shaker (New Brunswick Scientific, Edison, N.J., USA) shaking at 225 rpm. Each of these cultures (mean OD₆₂₅˜1.4 or 1.43×10⁸ CFU/ml) was transferred to a 0.45 μm nitrocellulose membrane filter (Millipore, Bedford, Mass. USA). Membranes were placed cell-side-up onto Mueller-Hinton agar containing 0.25 μg CIP/ml and incubated for ˜40 h. Cells from a single colony from each positive plate were streaked onto blood agar and grown overnight at 35° C. Cells from these plates were used to prepare frozen stocks and to isolate DNA for sequencing (see below). The most common unique genotype, S1-1, was subjected to a subsequent round of selection on Mueller-Hinton agar containing 1.5 μg CIP/ml. Likewise, the most common genotype from this selection, S2-1, was subjected to a third and final selection on agar containing 24 μg CIP/ml.

Mutation rates. Mutation rates for steps 1, 2 and 3 ciprofloxacin resistant mutants were determined using 96 independent cultures of the wild type Ames −/−, S1-1, and S2-1, respectively. A single colony of the starting isolate was suspended in LB broth and used to inoculate each of the independent cultures with approximately 1,000 cells. For steps 1 and 3 mutants, 96 1 ml cultures were grown in LB broth in four 24-well plates (Costar). For step 2, 96 100 μI were grown in LB broth cultures in a single 96-well plate (Costar). All plates were incubated overnight at 37° C. in a G24 Environmental Incubator Shaker (New Brunswick Scientific, Edison, N.J., USA) shaking at 225 rpm. Six cultures were chosen at random for each step and used to determine the average total number of cells present in each culture. The remaining 90 cultures were plated onto Mueller-Hinton ciprofloxacin plates with concentrations of 0.25 μg CIP/ml, 1.5 μg CIP/ml, and 24 μg CIP/ml for steps 1, 2, and 3, respectively. For step 2, the 100 μl cultures were directly plated. For steps 1 and 3, the 1 ml cultures were transferred to sterile 1.5 ml microcentrifuge tubes and centrifuged at 3,000×g for 5 min. Approximately 850 μl of the supernatant was removed, the pellet was resuspended in the remaining broth and plated. All of the plates were incubated at 37° C. for ˜48 h. Up to four putative resistant colonies from each positive plate were transferred to fresh selective medium, and incubated at 37° C. for ˜48 h to confirm resistance. The number of plates devoid of resistant mutants represents zero mutational events. This value was used with the cell count in the Poisson distribution to estimate the mutation rate for each step.

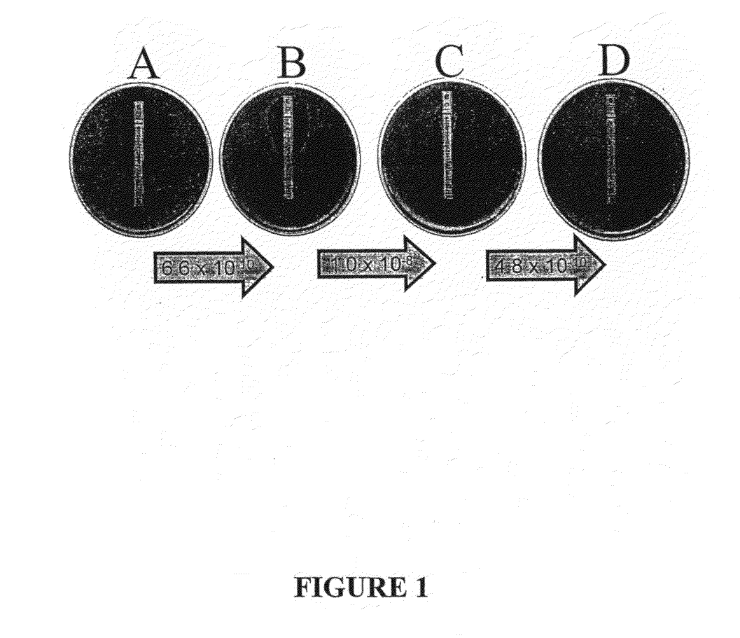

Susceptibility testing. MICs were determined by the Mueller-Hinton agar dilution method according to the guidelines of the National Committee for Clinical Laboratory Standards (National committee, 1997). The E-test strips (AB BIODISK) were used for rapid screening and are shown in FIG. 1.

### REFERENCES

1. Ahmed, M., L. Lyass, P. N. Markham, S. S. Taylor, N. Vazquez-Laslop, and A. A. Neyfakh. 1995. Two highly similar multidrug transporters of Bacillus subtilis whose expression is differentially regulated. J. Bacteriol, 177:3904-3910.
2. Ball, P. 1994. Bacterial resistance to fluoroquinolones: lessons to be learned. Infection. 2:S140-S147.
3. Berends, B. E., A. E. van den Bogaard, F. Van Knapen, and J. M. Snijders. 2001. Human health hazards associated with the administration of antimicrobials to slaughter animals. Part II. An assessment of the risks of resistant bacteria in pigs and pork. Vet. Q. 23:10-21.
4. Brook I., T. B. Elliott, H. I. Pryor 2nd, T. E. Sautter, B. T. Gnade, J. H. Thakar, G. B. Knudson. 2001. In vitro resistance of Bacillus anthracis Sterne to doxycyclin, macrolides and quinolones. Int. J. Antimicrob Agents. 18:559-562.
5. Brunner, M., U. Hollenstein, S. Delacher, D. Jager, R. Schmid, E. Lackner, A. Georgopoulos, H.G. Eichler, M. Muller. 1999. Distribution and antimicrobial activity of ciprofloxacin in human soft tissues. Antimicrob. Agents. Chemother. 43:1307-1309.
6. Caprioli, A., L. Busani, J. L. Martel, and R. Helmuth. 2000 Monitoring of antibiotic resistance in bacteria of animal origin: epidemiological and microbiological methodologies. Int. J. Antimicrob. Agents, 14:295-301.
7. Charvalos, E., Y. Tselentis, M. M. Hamzehpour, T. Kohler, and J. C. Pechere. 1995. Evidence for an efflux pump in multidrug-resistant Campylobacter jejuni. Antimicrob. Agents. Chemother. 39:2019-2022.
8. Choe, C. H., S. S. Bouhaouala, I. Brook, T. B. Elliot, and G. B. Knudson. 2000. In vitro development of resistance to ofloxacin and doxycycline in Bacillus anthracis Sterne. Antimicrob. Agents. Chemother. 44:1766,
9. Dong Y., X. Zhao, J. Domagala, and K. Drlica. 1999. Effect of fluoroquinolone concentration on selection of resistant mutants of Mycobacterium bovis BCG and Staphylococcus aureus. Antimicrob. Agents. Chemother. 43:1756-1758.
10. Drlica, K. and X. Zhao. 1997. DNA gyrase, topoisomerase IV, and the 4-quinolones. Microbiol. Mol. Biol. Rev. 61:377-392.
11. Endtz, H. P., G. J. Ruijs, B. van Klingeren, W. H. Jansen, T. van der Reyden, and R. P. Mouton. 1991. Quinolone resistance in campylobacter isolated from man and poultry following the introduction of fluoroquinolones in veterinary medicine. J Antimicrob. Chemother. 27:199-208.
12. Ferrero, L., B. Cameron, and J. Crouzet. 1995. Analysis of gyrA and grlA mutations in stepwise-selected ciprofloxacin-resistant mutants of Staphylococcus aureus. Antimicrob. Agents Chemother. 39:1554-1558.
13. Ferrero, L., B. Cameron, B. Manse, D. Lagneaux, J. Crouzet, A. Framechon, and F. Blanche. 1994. Cloning and primary structure of Staphylococcus aureus DNA topoisomerase IV: a primary target of fluoroquinolones. Mol. Microbiol. 13:641-653.
14. Friedlander, A, M. 1999. Clinical aspects, diagnosis and treatment of anthrax. J. Appl. Microbiol. 87:303.
15. Friedlander, A. M., S. L. Welkos, M. L. Pitt, J. W. Ezzell, P. L. Worsham, K. J. Rose, B. E. Ivins, J. R. Lowe, G. B. Howe, P. Mikesell P, et al. 1993. Postexposure prophylaxis against experimental inhalation anthrax. J. Infect. Dis. 167:1239-1243
16. Harrell, L. J., G. L. Andersen, and K. H. Wilson. 1995. Genetic variability of Bacillus anthracis and related species. J. Clin. Microbiol. 33:1847-1850.
17. Hugh-Jones, M. 1999. 1996-97 Global Anthrax Report. J. Appl. Microbiol. 87:189-191.
18. Ivins B. E., J. W. Ezzell, J. Jemski, K. W. Hedlund, J. D. Ristroph, S. H. Leppla. 1986 Immunization studies with attenuated strains of Bacillus anthracis. Infect. Immun. 1986 52:454-458.
19. Keim, P., A, Kalif, J. Schupp, K. Hill, S. E. Travis, K. Richmond, D. M. Adair, M. Hugh-Jones, C. R. Kuske, and P. Jackson. 1997. Molecular evolution and diversity in Bacillus anthracis as detected by amplified fragment length polymorphism markers. J. Bacteriol. 179:818-824.
20. Keim, P., L. B. Price, A. M. Klevytska, K. L. Smith, J. M. Schupp, R. Okinaka, P. J. Jackson, and M. E. Hugh-Joues. 2000. Multiple-locus variable-number tandem repeat analysis reveals genetic relationships within Bacillus anthracis. J. Bacteriol. 182:2928-2936.
21. Neyfakh, A. A. 1992. The multidrug efflux transporter of Bacillus subtilis is a structural and functional homolog of the Staphylococcus NorA protein. Antimicrob. Agents Chemother. 36:484-485.
22. Neyfakh, A. A., Borsch, C. M., and Kaatz, G. W. 1993. Fluoroquinolone resistance protein NorA of Staphylococcus aureus is a multidrug efflux transporter. Antimicrob. Agents. Chemother. 37:128-129.
23. Ng, E. Y., M. Trucksis, and D. C. Hooper. 1996. Quinolone resistance mutations in topoisomerase IV: relationship to the flqA locus and genetic evidence that topolsomerase IV is the primary target and DNA gyrase is the secondary target of fluoroquinolones in Staphylococcus aureus. Antimicrob Agents Chemother. 40:1881-1888.
24. Odendaal, M. W., P. M. Pieterson, V. de Vos, and A. D. Botha. 1991. The antibiotic sensitivity patterns of Bacillus anthracis isolated from the Kruger National Park. Onderstepoort J. Vet. Res. 58:17-9.
25. Pan and Fisher, X. S. and L. M. Fisher. 1998. DNA gyrase and topoisomerase IV are dual targets of clinafloxacin action in Streptococcus pneumoniae. Antimicrob. Agents Chemother. 42:2810-2816.
26. Patra, G., J. Vaissaire, M. Weber-Levy, C. Le Doujet, and M. Mock. 1998. Molecular characterization of Bacillus strains involved in outbreaks of anthrax in France in 1997. J. Clin. Microbiol 36:3412-3414.
27. Piddock, L. J. 1999. Mechanisms of fluoroquinolone resistance: an update 1994-1998. Drugs. 58:11-18.
28. Pomerantsev, A. P., N. A. Shishkova, and L. I. Marinin. 1992. [Comparison of therapeutic effects of antibiotics of the tetracycline group in the treatment of anthrax caused by a strain inheriting tet-gene of plasmid pBC16]. Antibiot. Khimioter. 37:31-34.
29. Price, L. B., M. Hugh-Jones, P. J. Jackson, and P. Kelm. Genetic diversity in the protective antigen gene of Bacillus anthracis. J. Bacteriol. 181:2358-2362.
30. Schedletzky, H., B. Wiedemann, P. Heisig. 1999. The effect of moxifloxacin on its target topoisomerases from Escherichia coli and Staphylococcus aureus. J. Antimicrob. Chemother. 43:31-37.
31. Schwarz, S., C. Kehrenberg, T. R. Walsh. 2001. Use of antimicrobial agents in veterinary medicine and food animal production. Int. J. Antimicrob. Agents. 17:431-437.
32. Segreti, J., T. D. Gootz, L. J. Goodman, G. W. Parkhurst; J. P. Quinn, B. A. Martin, and G. M. Trenholme. 1992. High-level quinolone resistance in clinical isolates of Campylobacter jejuni. J. Infect. Dis. 165:667-670.
33. Tran J. H., Jacoby, G. A. 2002. Mechanism of plasmid-mediated quinolone resistance. Proc Natl Acad Sci USA. 99:5638-5642.
34. Trucksis, M., D. C. Hooper, J. S. Wolfson. 1.991. Emerging resistance to fluoroquinolones in staphylococci: an alert. Ann. Intern. Med, 114:424-426.
35. van Den Bogaard, A. E., N. London, C. Driessen, and E. E. Stobberingh. 2001. Antibiotic resistance of faecal Escherichia coli in poultry, poultry farmers and poultry slaughterers. J Antimicrob. Chemother. 47:763-771.
36. van den Bogaard, A. E. and E. E. Stobberingh. 2000. Epidemiology of resistance to antibiotics. Links between animals and humans. Int. J. Antimicrob. Agents. 14:327-35.
37. Vogler, A. J., J. D. Busch, S. Percy-Fine, C. M. Tipton-Hunton, K. L. Smith, P. Kelm. 2001. Molecular Analysis of Rifampicin Resistance in Bacillus anthracis and B. cereus. J Antimicrob. Chemother. 46:511-513.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

We claim:

1. An isolated oligonucleotide comprising at least 12 consecutive nucleotides of a nucleic acid sequence selected from the group of consisting of SEQ ID NO: 1, SEQ ID NO: 2; SEQ ID NO:3; SEQ ID NO: 4; SEQ ID NO: 5; SEQ ID NO: 6; SEQ ID NO: 7; SEQ ID NO: 8; SEQ ID NO: 9; SEQ ID NO: 10; SEQ ID NO: 11; SEQ ID NO: 12; SEQ ID NO: 13; SEQ ID NO: 14; SEQ ID NO: 15; SEQ ID NO: 16; SEQ ID NO: 17; SEQ ID NO: 18; SEQ ID NO: 19; SEQ ID NO: 20; SEQ ID NO: 21; SEQ ID NO: 22; SEQ ID NO: 23; SEQ ID NO: 24; SEQ ID NO: 25; SEQ ID NO: 26; SEQ ID NO: 27; SEQ ID NO: 28; SEQ ID NO: 29; SEQ ID NO: 30; SEQ ID NO: 31; SEQ ID NO: 32; SEQ ID NO: 33; SEQ ID NO: 34; SEQ ID NO: 35; SEQ ID NO: 36; SEQ ID NO: 37; SEQ ID NO: 38; SEQ ID NO: 39; SEQ ID NO: 40; SEQ ID NO: 41; SEQ ID NO: 42; SEQ ID NO: 43; SEQ ID NO: 44; SEQ ID NO: 45; SEQ ID NO: 46; SEQ ID NO: 47; SEQ ID NO: 48; SEQ ID NO: 49; SEQ ID NO: 50; SEQ ID NO: 51; SEQ ID NO: 52; and SEQ ID NO: 53; wherein the oligonucleotide is capable of binding selectively to DNA indicating fluoroquinoline resistance in Bacillus anthracis.

2. The oligonucleotide of claim 1 immobilized on a solid surface.

3. The oligonucleotide of claim 1, further comprising an observable marker.

4. The oligonucleotide of claim 3, wherein the observable marker is a fluorescent label.

5. The oligonucleotide of claim 3, wherein the observable marker is a radioactive group.

6. The oligonucleotide of claim 1, wherein the fluoroquinoline is ciprofloxacin.

7. A pair of oligonucleotide primers selected from the group of oligonucleotide pairs consisting of: SEQ ID NO: 1 and SEQ ID NO: 2; SEQ ID NO: 3 and SEQ ID NO: 4; SEQ ID NO: 5 and SEQ ID NO: 6; SEQ ID NO: 7 and SEQ ID NO: 8; SEQ ID NO: 9 and SEQ ID NO: 10; SEQ ID NO: 11 and SEQ ID NO: 12; SEQ ID NO: 13 and SEQ ID NO: 14; SEQ ID NO: 15 and SEQ ID NO: 16; SEQ ID NO: 17 and SEQ ID NO: 18; SEQ ID NO: 19 and SEQ ID NO: 20; SEQ ID NO: 21 and SEQ ID NO: 22; SEQ ID NO: 23 and SEQ ID NO: 24; SEQ ID NO: 25 and SEQ ID NO: 26; SEQ ID NO: 27 and SEQ ID NO: 28; SEQ ID NO: 29 and SEQ ID NO: 30; SEQ ID NO: 31 and SEQ ID NO: 32; SEQ ID NO: 33 and SEQ ID NO: 34; SEQ ID NO: 35 and SEQ ID NO: 36; SEQ ID NO: 37 and SEQ ID NO: 38; and SEQ ID NO: 39 and SEQ ID NO: 40; wherein the pair of oligonucleotide primers is capable of binding selectively to DNA indicating fluoroquinoline resistance in Bacillus anthracis.

8. The pair of oligonucleotides in claim 7, wherein the fluoroquinoline is ciprofloxacin.

9. An oligonucleotide primer selected from the group consisting of: SEQ ID NO: 41; SEQ ID NO: 42; SEQ ID NO: 43; SEQ ID NO: 44; SEQ ID NO: 45; SEQ ID NO: 46; SEQ ID NO: 47; SEQ ID NO: 48; SEQ ID NO: 49; SEQ ID NO: 50; SEQ ID NO: 51, SEQ ID NO: 52; and SEQ ID NO: 53, wherein the primer is capable of detecting a single nucleotide polymorphism, wherein the single nucleotide polymorphism is characteristic of fluoroquinoline resistance in Bacillus anthracis.

10. The oligonucleotide primer of claim 9, wherein the primer comprises a polynucleotide tail capable of producing a customized amplicon length.

11. A method for detecting fluoroquinoline resistance in Bacillus anthracis comprise the steps of
i. providing a DNA sample from a Bacillus anthracis;
ii. viding one or more primer pairs from claim 7;
iii. amplifying said DNA with said primer pair; and
iv. comparing a result of said amplification step with a result of amplification of a known fluoroquinoline resistant Bacillus anthracis with said primer pair.

12. The method of claim 11, wherein said amplification step further comprises multiplexing.

13. A method for detecting fluoroquinoline resistance in Bacillus anthracis comprising the steps of:
i. providing a DNA sample from Bacillus anthracis;
ii. viding one or more oligonucleotides from claim 1;
iii. combining said oligonucleotide and said DNA under conditions whereby said oligonucleotide binds to said DNA; and
iv. detecting the presence or absence of bound oligonucleotide,
wherein the presence of bound oligonucleotide indicates fluoroquinoline resistance in B. anthracis.

14. The method of claim 13, wherein said oligonucleotide comprises an observable marker.

15. The method of claim 14, wherein said observable marker is a fluorescent or radioactive group.

16. A method for detecting a fluoroquinoline resistance in Bacillus anthracis comprising the steps of
i. providing a DNA sample from a Bacillus anthracis;
ii. viding one or more primer pairs from claim 7;
iii. providing one or more primers from claim 9; and
iv. amplifying said DNA with said primer pairs and said primer;
v. comparing the results of said amplification step with results of amplification of a known fluoroquinoline resistant B. anthracis with said primers.

17. A kit for the molecular detection of fluoroquinoline resistance in Bacillus anthracis strain by amplification of DNA, said kit comprising:
one or more oligonucleotide primers from claim 1, wherein the oligonucleotide primer is capable of indicating fluoroquinoline resistance in Bacillus anthracis.

18. The kit of claim 17 further comprising dNTPs, taq polymerase, salts and buffers suitable for causing amplification of said DNA in a PCR instrument.

19. The kit of claim 18 wherein said dNTPs are labeled with a fluorescent or radioactive group.

20. A kit for molecular detection of fluoroquinoline resistance in Bacillus anthracis by assay of DNA, wherein the DNA is characteristic of a fluoroquinoline resistance, said kit comprising one or more primers from claim 9.

21. The kit of claim 20, wherein said primers are labeled with a fluorescent or radioactive group.

22. The kit of claim 21, further comprising salts and buffers suitable for causing binding of said DNA to said primers.


SEQUENCE LISTING (53 sequences)

SEQ ID NO: 1 - DNA, 35 bp, Artificial
  Bacillus anthracis
gtgttaggtgaccgctttgcacgttatagtaaata

SEQ ID NO: 2 - DNA, 31 bp, Artificial
  Bacillus anthracis
gtaaaacaaccggttcttcactcgtatcatc

SEQ ID NO: 3 - DNA, 45 bp, Artificial
  Bacillus anthracis
acgtattaattccatagagattttagacattcttgcttctgtata

SEQ ID NO: 4 - DNA, 37 bp, Artificial
  Bacillus anthracis
catttttagattacgcaatgagtgttatcgtatctcg

SEQ ID NO: 5 - DNA, 28 bp, Artificial
  Bacillus anthracis
ggtacgacagttgcccaaaatgatggtt

SEQ ID NO: 6 - DNA, 22 bp, Artificial
  Bacillus anthracis
caagcggaagcaattgtatcct

SEQ ID NO: 7 - DNA, 35 bp, Artificial
  Bacillus anthracis
cgcgtcgatcatcactatatgttttcttaactctc

SEQ ID NO: 8 - DNA, 29 bp, Artificial
  Bacillus anthracis
attattattcgcgggaaagcagaggttga

SEQ ID NO: 9 - DNA, 30 bp, Artificial
  Bacillus anthracis
gtctcatcacgtacttcagcaatgccatct

SEQ ID NO: 10 - DNA, 32 bp, Artificial
  Bacillus anthracis
tcggctaaaacagtcggtaacgttattggtaa

SEQ ID NO: 11 - DNA, 17 bp, Artificial
  Bacillus anthracis
cggatccccgtcaacac

SEQ ID NO: 12 - DNA, 30 bp, Artificial
  Bacillus anthracis
cggatcaattatgggaaacaacgatgaatc

SEQ ID NO: 13 - DNA, 27 bp, Artificial
  Bacillus anthracis
aagcgggaggtcatgaaacttctctgc

SEQ ID NO: 14 - DNA, 29 bp, Artificial
  Bacillus anthracis
agtggtaagttaacacccgcacaatcacg

SEQ ID NO: 15 - DNA, 20 bp, Artificial
  Bacillus anthracis
cccttgtttcgcagaaccac

SEQ ID NO: 16 - DNA, 20 bp, Artificial
  Bacillus anthracis
ttgaagctttcgtttcctat

SEQ ID NO: 17 - DNA, 31 bp, Artificial
  Bacillus anthracis
ctaattctgcttcaatcccattttgttcacc

SEQ ID NO: 18 - DNA, 28 bp, Artificial
  Bacillus anthracis
tagcgttatagataaagggcgaggaatg

SEQ ID NO: 19 - DNA, 26 bp, Artificial
  Bacillus anthracis
acaccgccattttcaaagcgttgttc

SEQ ID NO: 20 - DNA, 30 bp, Artificial
  Bacillus anthracis
gattttggattaggaaaggggcaaggagtt

SEQ ID NO: 21 - DNA, 22 bp, Artificial
  Bacillus anthracis
cgacggaattgaacacgaaaca

SEQ ID NO: 22 - DNA, 18 bp, Artificial
  Bacillus anthracis
tacagatgccccaacacc

SEQ ID NO: 23 - DNA, 33 bp, Artificial
  Bacillus anthracis
atgggacgtcctgctgtagaagttattatgacc

SEQ ID NO: 24 - DNA, 32 bp, Artificial
  Bacillus anthracis
agttaaaccttcacgaacgtcctcaccagtta

SEQ ID NO: 25 - DNA, 35 bp, Artificial
  Bacillus anthracis
acgtatgaaggtggaacacatgaagtaagggttta

SEQ ID NO: 26 - DNA, 31 bp, Artificial
  Bacillus anthracis
gctttctcaatatcaaaatctccgccaatgt

SEQ ID NO: 27 - DNA, 29 bp, Artificial
  Bacillus anthracis
cgtcacttccaagcgattttaccactgaa

SEQ ID NO: 28 - DNA, 39 bp, Artificial
  Bacillus anthracis
acctcctcttacatttccgttacacatacattgatttat

SEQ ID NO: 29 - DNA, 29 bp, Artificial
  Bacillus anthracis
gggggataaagtagagccacgtcgtaact

SEQ ID NO: 30 - DNA, 20 bp, Artificial
  Bacillus anthracis
aggaaaacgcgctggtaaca

SEQ ID NO: 31 - DNA, 31 bp, Artificial
  Bacillus anthracis
cagcaatgcgttatacagaagcaagaatgtc

SEQ ID NO: 32 - DNA, 25 bp, Artificial
  Bacillus anthracis
tgccttttcaagttcataagcagta

SEQ ID NO: 33 - DNA, 29 bp, Artificial
  Bacillus anthracis
ggaagtacgtcgtgatgccaatgctaatg

SEQ ID NO: 34 - DNA, 35 bp, Artificial
  Bacillus anthracis
atacctttcgctgtacgactatactctgggatttc

SEQ ID NO: 35 - DNA, 34 bp, Artificial
  Bacillus anthracis
cagaacaaaacatcgccattacgttaactcataa

SEQ ID NO: 36 - DNA, 36 bp, Artificial
  Bacillus anthracis
agagatttgatcaactggcatacgaataataacacc

SEQ ID NO: 37 - DNA, 22 bp, Artificial
  Bacillus anthracis
tcagcacgtattgttggtgaag

SEQ ID NO: 38 - DNA, 23 bp, Artificial
  Bacillus anthracis
tgcccatccaacaagcatataac

SEQ ID NO: 39 - DNA, 19 bp, Artificial
  Bacillus anthracis
aaagcgttccgtaagtcgg

SEQ ID NO: 40 - DNA, 26 bp, Artificial
  Bacillus anthracis
ttattaccatgcatctcaactaaaac

SEQ ID NO: 41 - DNA, 21 bp, Artificial
  Bacillus anthracis
atcggtaagtatcaccctcat

SEQ ID NO: 42 - DNA, 25 bp, Artificial
  Bacillus anthracis
nnnnncggtaagtatcaccctcatg

SEQ ID NO: 43 - DNA, 29 bp, Artificial
  Bacillus anthracis
nnnnnnnnggtaagtatcaccctcatggt

SEQ ID NO: 44 - DNA, 33 bp, Artificial
  Bacillus anthracis
nnnnnnnnnnnnnccatcgtttcataaacagct

SEQ ID NO: 45 - DNA, 33 bp, Artificial
  Bacillus anthracis
nnnnnnnnnnnnnccatcgttgcataaacagct

SEQ ID NO: 46 - DNA, 33 bp, Artificial
  Bacillus anthracis
nnnnnnnnnnnnnccatcgttttataaacagct

SEQ ID NO: 47 - DNA, 33 bp, Artificial
  Bacillus anthracis
nnnnnnnnnnnnnccatcgttgtataaacagct

SEQ ID NO: 48 - DNA, 37 bp, Artificial
  Bacillus anthracis
nnnnnnnnnnnnnnnnnngccatacgtaccatcgttt

SEQ ID NO: 49 - DNA, 37 bp, Artificial
  Bacillus anthracis
nnnnnnnnnnnnnnnnnngccatacgtaccatcgttg

SEQ ID NO: 50 - DNA, 41 bp, Artificial
  Bacillus anthracis
nnnnnnnnnnnnnnnnnnnnnncgccatacgtaccatcgtt

SEQ ID NO: 51 - DNA, 45 bp, Artificial
  Bacillus anthracis
nnnnnnnnnnnnnnnnnnnnnnnnnnnnncacccgcacggtgatt

SEQ ID NO: 52 - DNA, 49 bp, Artificial
  Bacillus anthracis
nnnnnnnnnnnnnnnnnnnnnnnnnnnngacttaaacgtaccatcgctt

SEQ ID NO: 53 - DNA, 53 bp, Artificial
  Bacillus anthracis
nnnnnnnnnnnnnnnnnnnnnnnnnnnnnnnnnagccatggtacgtttaagtc